(19) United States
(12) Patent Application Publication
(10) Pub. No.: US 2024/0013431 A1
(43) Pub. Date: Jan. 11, 2024

(54) IMAGE CAPTURE DEVICES, SYSTEMS, AND METHODS

(71) Applicant: Warby Parker Inc., New York, NY (US)

(72) Inventors: David H. Goldberg, New York, NY (US); David J. DeShazer, Verona, NJ (US); Kathleen Maloney, New York, NY (US)

(73) Assignee: Warby Parker Inc., New York, NY (US)

(21) Appl. No.: 18/346,948

(22) Filed: Jul. 5, 2023

Related U.S. Application Data
(60) Provisional application No. 63/359,311, filed on Jul. 8, 2022.

(51) Int. Cl. G06T 7/70; G06T 7/20; G06T 7/62; G06V 40/18; H04N 23/695

(52) U.S. Cl. CPC G06T 7/70; G06T 7/20; G06T 7/62; G06V 40/193; H04N 23/695; G06V 2201/07; G06T 2207/20084

(57) ABSTRACT

A system may include an image capture device a processor communicatively coupled to the image capture device. The image capture device may be configured to obtain one or more images. The processor may be configured to receive a first image obtained by the image capture device, determine if a first object is in the first image, determine if a size and a location of the first object in the first image meet threshold values if the first object is determined to be in the first image, determine at least one corrective action to be taken if at least one of the size and the location of the first object in the image does not meet the threshold values, and cause an instruction for taking the at least one corrective action to be communicated to a user. Methods and machine-readable storage media also are disclosed.

### CROSS-REFERENCE TO RELATED APPLICATION

This application claims priority to U.S. Provisional Patent Application No. 63/359,311, filed Jul. 8, 2022, the entirety of which is incorporated by reference herein.

### FIELD OF DISCLOSURE

The disclosed devices, systems, and methods relate to image capturing and processing. More specifically, the disclosed devices, systems, and methods relate to image capturing and processing that may be used for patient care and/or telehealth.

### BACKGROUND

Consumer cameras and other image capture devices continue to improve and now are able to obtain near cinematic quality images and videos. Today, these cameras and image capture devices are either incorporated in or configured to work with cellular phones, laptops, desktop computers, and other computing devices. While these consumer devices are typically used for social reasons (e.g., taking pictures and/or video of people, places, and things to share with others), they have the capability to be used for diagnostic reasons.

### SUMMARY

In some aspects, a system may include an image capture device and a processor communicatively coupled to the image capture device. The image capture device may be configured to obtain one or more images. The processor may be configured to receive a first image obtained by the image capture device, determine if a first object is in the first image, determine if a size and a location of the first object in the first image meet threshold values if the first object is determined to be in the first image, determine at least one corrective action to be taken if at least one of the size and the location of the first object in the image does not meet the threshold values, and cause an instruction for taking the at least one corrective action to be communicated to a user.

In some aspects, the first object may be at least one of an iris and a pupil of an eye.

In some aspects, the first object may be a contact lens.

In some aspects, the at least one corrective action may include moving the image capture device in at least one direction relative to at least one of a user and the object.

In some aspects, the image capture device may be a camera located on a side of a mobile device that is opposite a side on which a screen is disposed.

In some aspects, the at least one corrective action may include moving an eye of a user relative to the image capture device.

In some aspects, the processor may be configured to determine if the first object is in the first image using a first neural network.

In some aspects, the processor may be configured to track the first object in at least one second image obtained from the image capture device using a second neural network that is different from the first neural network.

In some aspects, the processor may be configured to store the first image in a non-transient machine-readable storage medium that is communicatively coupled to the processor if the size and the location of the first object in the first image meet threshold values.

In some aspects, the processor may be configured to analyze a plurality of images received from the image capture device and determine a blink of an eye.

In some aspects, the instruction for taking at least one corrective action may be communicated to the user by an audio output device that is communicatively coupled to the processor.

In some aspects, the instruction for taking at least one corrective action may be communicated to the user haptically.

In some aspects, the instruction for taking at least one corrective action may be presented to the user on a display that is communicatively coupled to the processor.

In some aspects, the processor may be configured to provide an indication that the size and the location of the first object in the image meets the threshold values.

In some aspects, the indication may include a haptic indication.

In some aspects, the indication may include a visual indication.

In some aspects, the indication may include an audible indication.

In some aspects, the indication may include an instruction to acquire a second image.

In some aspects, a method may include determining, by a first processing module, if an object is present in a first image received from an image capture device; if the object is determined to be present in the first image, then determining, by a second processing module, if the object is meets at least one predetermined criterion; if the object does not meet the at least one predetermined criterion, then determining, by a third processing module, at least one adjustment to be made; and communicating an instruction to perform the at least one adjustment. The at least one predetermined criterion may include at least one of a size and a location in an image.

In some aspects, the first, second, and third processors may be the same processor.

In some aspects, the first, second, and third processors may be different processors.

In some aspects, the instruction to perform the at least one adjustment may be communicated to a user using an audio output device.

In some aspects, the instruction to perform the at least one adjustment may be communicated to a user haptically.

In some aspects, the instruction to perform the at least one adjustment may be displayed to a user.

In some aspects, the method may include providing an indication that the at least one predetermined criterion has been met.

In some aspects, the indication may include a haptic indication.

In some aspects, the indication may include a visual indication.

In some aspects, the indication may include an audible indication.

In some aspects, the indication may include an instruction to acquire a second image.

In some aspects, a machine-readable storage medium may store executable code. The executable code, when executed by a processor, may cause the processor to perform a method. The method may include receiving a first image obtained by an image capture device; determining if a first object is in the first image; determining if a size and a location of the first object in the first image meet threshold values if the first object is determined to be in the first image; determining at least one corrective action to be taken if at least one of the size and the location of the first object in the image does not meet the threshold values; and causing an instruction for taking the at least one corrective action to be communicated to a user.

In some aspects, the first object may be at least one of an iris and a pupil of an eye.

In some aspects, the first object may be a contact lens.

In some aspects, the at least one corrective action may include moving the image capture device in at least one direction relative to at least one of an eye of a user and the object.

In some aspects, the image capture device may be a camera located on a side of a mobile device that is opposite a side on which a screen is disposed.

In some aspects, the at least one corrective action may include moving an eye of a user relative to the image capture device.

In some aspects, determining if the first object is in the first image may include using a first neural network.

In some aspects, the method may include tracking the first object in at least one second image obtained from the image capture device using a second neural network that is different from the first neural network.

In some aspects, the method may include storing the first image in a non-transient machine-readable storage medium that is communicatively coupled to the processor if the size and the location of the first object in the first image meet threshold values.

In some aspects, the method may include analyzing a plurality of images received from the image capture device and determine a blink of an eye.

In some aspects, the instruction for taking at least one corrective action may be communicated to the user by an audio output device.

In some aspects, the instruction for taking at least one corrective action may be communicated to the user haptically.

In some aspects, the instruction for taking at least one corrective action may be presented to the user on a display.

In some aspects, the method may include providing an indication that the at least one of the size and the location of the first object in the image meets the threshold values.

In some aspects, the indication may include a haptic indication.

In some aspects, the indication may include a visual indication.

In some aspects, the indication may include an audible indication.

In some aspects, the indication may include an instruction to acquire a second image.

In some aspects, a system may include an image capture device and a processor communicatively coupled to the image capture device. The image capture device may be configured to obtain one or more images. The processor may be configured to receive a first image obtained by the image capture device; determine if a first object is in the first image; determine if a size and a location of the first object in the first image meet threshold values if the first object is determined to be in the first image; cause an indication that the first image meets the threshold values to be communicated to a user.

In some aspects, the processor may be configured to determine at least one corrective action to be taken if at least one of the size and the location of the first object in the image does not meet the threshold values and cause an instruction for taking the at least one corrective action to be communicated to the user.

In some aspects, a method may include determining, by a first processing module, if an object is present in a first image received from an image capture device; if the object is determined to be present in the first image, then determining, by a second processing module, if the object is meets at least one predetermined criterion; and if the object meets the at least one predetermined criterion, then providing an indication to a user. The at least one predetermined criterion may include at least one of a size and a location in an image.

In some aspects, the method may include if the object does not meet the at least one predetermined criterion, then determining, by a third processing module, at least one adjustment to be made; and communicating an instruction to perform the at least one adjustment.

In some aspects, a machine-readable storage medium may store executable code. The executable code, when executed by a processor, may cause the processor to perform a method. The method may include receiving a first image obtained by an image capture device; determining if a first object is in the first image; determining if a size and a location of the first object in the first image meet threshold values if the first object is determined to be in the first image; and causing an indication that the first image meets the threshold values to be communicated to a user.

In some aspects, the method may include determining at least one corrective action to be taken if at least one of the size and the location of the first object in the image does not meet the threshold values and causing an instruction for taking the at least one corrective action to be communicated to a user.

### BRIEF DESCRIPTION OF THE DRAWINGS

FIG. 1 is a schematic diagram of one example of a system in accordance with some embodiments;

FIG. 2 is a functional block diagram of one example of a computing device that may be used with the system illustrated in FIG. 1 in accordance with some embodiments;

FIG. 3 is a functional block diagram of a processing pipeline that may be implemented by a computing device in accordance with some embodiments;

FIGS. 3A and 3B illustrate examples of a front and a back of a computing device, respectively, in accordance with some embodiments;

FIGS. 4A and 4B illustrate examples of a number of selections that a provider user and a patient user may be able to make via an app and/or web portal, respectively, in accordance with some embodiments;

FIG. 5 illustrates one example of a process flow that may be performed to obtain one or more images and/or videos in accordance with some embodiments;

FIG. 6A illustrates one example of an image or frame of an eye having an iris and a pupil in accordance with some embodiments;

FIG. 6B illustrates one example of an image of an eye in which an edge of a pupil and an edge of a contact lens are highlighted in accordance with some embodiments; and

FIG. 6C illustrates one example of an image of an eye in which an edge of a contact lens is highlighted in accordance with some embodiments.

### DETAILED DESCRIPTION

This description of the exemplary embodiments is intended to be read in connection with the accompanying drawings, which are to be considered part of the entire written description.

The disclosed systems and methods advantageously facilitate remote medical examinations (e.g., telehealth). In some embodiments, the medical examinations include medical examinations that relate to the eye. Examples of conditions that may be assess may include, but are not limited to, lid lesions, swelling, ptosis, redness/swelling of conjunctiva, dry eye (e.g., capped glands and cornea evaluation), pterygium/pinguecula, contact placement, fit, and irritation, glaucoma, and cataracts, to list only a few possibilities. Although the descriptions herein relate to an app that images the eye to facilitate telehealth, it should be understood that other types of medical or diagnostic evaluations may be performed, such as those concerning the ear, nose, throat, and other body parts.

In some embodiments, the disclosed systems and methods may leverage imaging devices available to a wide variety of users, such as those provided on a mobile device (e.g., an Apple iPhone, Apple iPad, Apple iPod, Samsung Galaxy), and may provide a user with audio, visual, and/or tactile instructions to facilitate the acquisition of one or more images that are useful in an eye examination. As described herein, the one or more images may include a single static image acquisition or it may include a series of images that comprise a video.

As described in greater detail below, the disclosed systems, devices, and methods may be implemented over networks such as, for example, the Internet. The Internet is a worldwide system of computer networks—a network of networks in which a user at one computer, terminal, or other device connected to the network can obtain information from any other computer, terminal, or device and communicate with users of other computers or devices. The most widely used part of the Internet is the World Wide Web (often-abbreviated “WWW” or called “the Web”).

One of the features of the Web is the use of hypertext, which is a method of cross-referencing. In most Web sites, certain words or phrases appear in text of a different color than the surrounding text. This text is often also underlined. Sometimes, there are hot spots, such as buttons, images, or portions of images that are “clickable.” Clicking on hypertext or a hot spot causes the downloading of another web page via a protocol such as hypertext transport protocol (HTTP). Using the Web provides access to millions of pages of information. Web “surfing” is done with a Web browser such as, for example, Apple Safari, Microsoft Edge, Mozilla Firefox, and Google Chrome. The appearance of a particular website may vary slightly depending on the particular browser used. Versions of browsers have “plug-ins,” which may provide animation, virtual reality, sound, and music. Interpreted programs (e.g., applets) may be run within the browser.

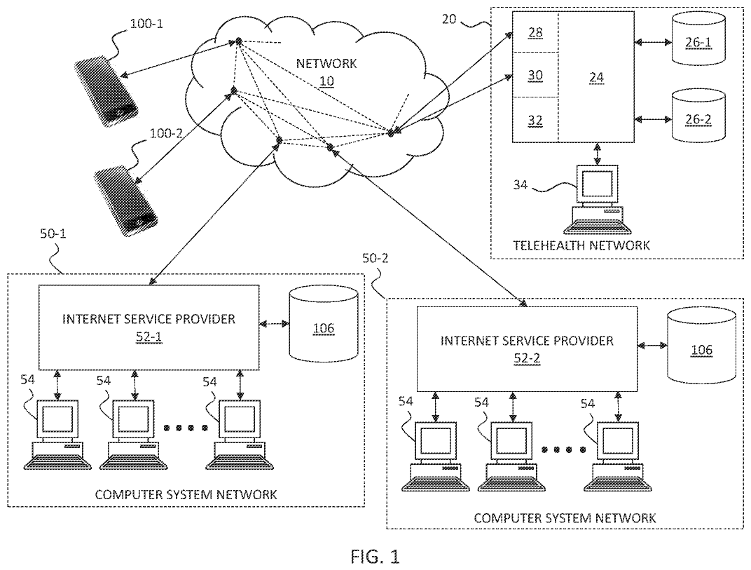

FIG. 1 shows a system in which a plurality of wireless devices 100-1 and 100-2 (collectively “wireless devices 100” or “mobile devices 100”) are connected via network 10 to one or more computer system networks 50-1, 50-2 (collectively “computer system networks 50”) and to telehealth network 20. Network 10 may be a wide area network (“WAN”), a local area network (“LAN”), personal area network (“PAN”), or the like. In one embodiment, network 10 is the Internet and mobile devices 100 are online. “Online” may mean connecting to or accessing source data or information from a location remote from other devices or networks coupled to network 10.

Telehealth network 20 may include one or more processing units 24 coupled to one or more data storage units 26-1, 26-2 (collectively referred to as “data storage units 26”). The processing unit 24 may provide front-end graphical user interfaces (“GUIs”), e.g., a patient GUI or interface 28, a provider GUI or portal 30, and a back-end or administrative GUI or portal 32 to a remote computer 54 or to local computer 34. The GUIs can take the form of, for example, a webpage that is displayed using a browser program local to remote computers 54 or to one or more local computers 34. It is understood that the telehealth network 20 may be implemented on one or more computers, servers, or other computing devices. For example, physical training network 20 may include servers programmed or partitioned based on permitted access to data stored in data storage units 26. Front- and back-end GUIs 28, 30, 32 may be portal pages that include various content retrieved from the one or more data storage devices 26. As used herein, a “portal” is not limited to general-purpose Internet portals or search engine, such as GOOGLE, but also includes GUIs that may be of interest to specific, limited audiences and that may provide the party access to a plurality of different kinds of related or unrelated information, links and tools as described below. “Webpage” and “website” may be used interchangeably herein.

Remote computers 54 may be part of a computer system network 50 and gain access to network 10 through an Internet service provider (“ISP”) 52-1, 52-2 (“ISPs 52”). Mobile devices 100 may gain access to network 10 through a wireless cellular communication network, a WAN hotspot, or through a wired or wireless connection with a computer as will be understood by one skilled in the art. Patients or providers may use remote computers 54 to gain access to the telehealth network 20.

In some embodiments, a mobile or computing device 100 includes any mobile or computing device capable of transmitting and receiving wireless signals. Examples of mobile instruments include, but are not limited to, mobile or cellular phones, personal digital assistants (“PDAs”), laptop computers, tablet computers, music players, and e-readers, to name only a few possible devices.

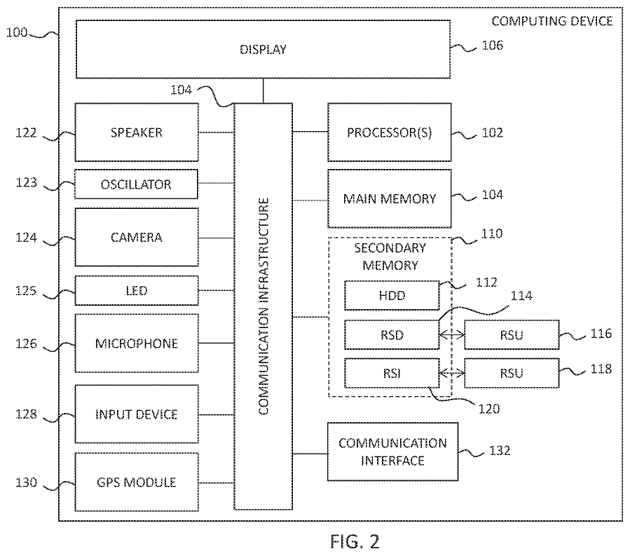

FIG. 2 is a block diagram of one example of an architecture of mobile device 100. As shown in FIG. 2, mobile device 100 may include one or more processors, such as processor(s) 102. Processor(s) 102 may be any central processing unit (“CPU”), microprocessor, micro-controller, or computational device or circuit for executing instructions. Processor(s) may be connected to a communication infrastructure 104 (e.g., a communications bus, crossover bar, or network). Various software embodiments are described in terms of this exemplary mobile device 100. After reading this description, it will be apparent to one of ordinary skill in the art how to implement the method using mobile devices 100 that may include other systems or architectures. One of ordinary skill in the art will understand that computers 34, 54 may have a similar and/or identical architecture as that of mobile devices 100. Put another way, computers 34, 54 can include some, all, or additional functional components as those of the mobile device 100 illustrated in FIG. 2.

Mobile device 100 may include a display 106 that displays graphics, video, text, and other data received from the communication infrastructure 104 (or from a frame buffer not shown) to a user (e.g., a patient, provider user, back-end user, or other user). Examples of such displays 106 include, but are not limited to, LCD screens, LED display, OLED display, touch screen (e.g., capacitive, resistive optical imaging, infrared), and a plasma display, to name a few possible displays. Mobile instrument 100 also may include a main memory 108, such as a random access (“RAM”) memory, and may also include a secondary memory 110. Secondary memory 110 may include a more persistent memory such as, for example, a hard disk drive (“HDD”) 112 and/or removable storage drive (“RSD”) 114, representing a magnetic tape drive, an optical disk drive, solid-state drive (“SDD”), or the like. In some embodiments, removable storage drive 114 may read from and/or writes to a removable storage unit (“RSU”) 116 in a manner that is understood by one of ordinary skill in the art. Removable storage unit 116 may represent a magnetic tape, optical disk, or the like, which may be read by and written to by removable storage drive 114. As will be understood by one of ordinary skill in the art, the removable storage unit 116 may include a tangible and non-transient machine-readable storage medium having stored therein computer software and/or data.

In some embodiments, secondary memory 110 may include other devices for allowing computer programs or other instructions to be loaded into mobile device 100. Such devices may include, for example, a removable storage unit (“RSU”) 118 and a corresponding interface (“RSI”) 120. Examples of such units 118 and interfaces 120 may include a removable memory chip (such as an erasable programmable read only memory (“EPROM”)), programmable read only memory (“PROM”)), secure digital (“SD”) card and associated socket, and other removable storage units 118 and interfaces 120, which allow software and data to be transferred from the removable storage unit 118 to mobile device 100.

Mobile device 100 may also include a speaker 122, an oscillator 123, a camera (or other image capture device) 124, a light emitting diode (“LED”) 125, a microphone 126, an input device 128, and a global positioning system (“GPS”) module 130. Examples of input device 128 include, but are not limited to, a keyboard, buttons, a trackball, or any other interface or device through which a user may input data. In some embodiments, input device 128 and display 106 are integrated into the same component or device. For example, display 106 and input device 128 may be touchscreen through which a user uses a finger, pen, and/or stylus to input data into mobile device 100.

Mobile device 100 also may include one or more communication interfaces 132, which allows software and data to be transferred between mobile device 100 and external devices such as, for example, another mobile device 100, a computer 34, 54, telehealth network and other devices that may be locally or remotely connected to mobile device 100. Examples of the one or more communication interfaces 132 may include, but are not limited to, a modem, a network interface (such as an Ethernet card or wireless card), a communications port, a Personal Computer Memory Card International Association (“PCMCIA”) slot and card, one or more Personal Component Interconnect (“PCI”) Express slot and cards, or any combination thereof. The one or more communication interfaces 132 may also include a wireless interface configured for short-range communication, such as near field communication (“NFC”), Bluetooth, or other interface for communication via another wireless communication protocol. As briefly noted above, one of ordinary skill in the art will understand that computers 34, 54 20 may include some or all components of mobile device 100.

Software and data transferred via the one or more communications interfaces 132 may be in the form of signals, which may be electronic, electromagnetic, optical, or other signals capable of being received by communications interfaces 132. These signals may be provided to communications interface 132 via a communications path or channel. The channel may be implemented using wire or cable, fiber optics, a telephone line, a cellular link, a radio frequency (“RF”) link, or other communication channels. The terms “non-transient computer program medium” and “non-transient computer readable medium” refer to media such as removable storage units 116, 118, or a hard disk installed in hard disk drive 112. These computer program products provide software to mobile device 100. Computer programs (also referred to as “computer control logic”) may be stored in main memory 108 and/or secondary memory 110. Computer programs may also be received via the one or more communications interfaces 132. Such computer programs, when executed by a processor(s) 102, enable the mobile device 100 to perform the methods discussed herein.

In some embodiments, where the method is partially or entirely implemented using software, the software may be stored in a computer program product and loaded into mobile device 100 using removable storage drive 114, hard drive 112, and/or communications interface 132. The software, when executed by processor(s) 102, may cause the processor(s) 102 to perform the functions of the methods described herein. In some embodiments, the method may be implemented primarily in hardware using, for example, hardware components such as application specific integrated circuits (“ASICs”). Implementation of the hardware state machine so as to perform the functions described herein will be understood by persons skilled in the art. In some embodiments, the methods may be implemented using a combination of both hardware and software.

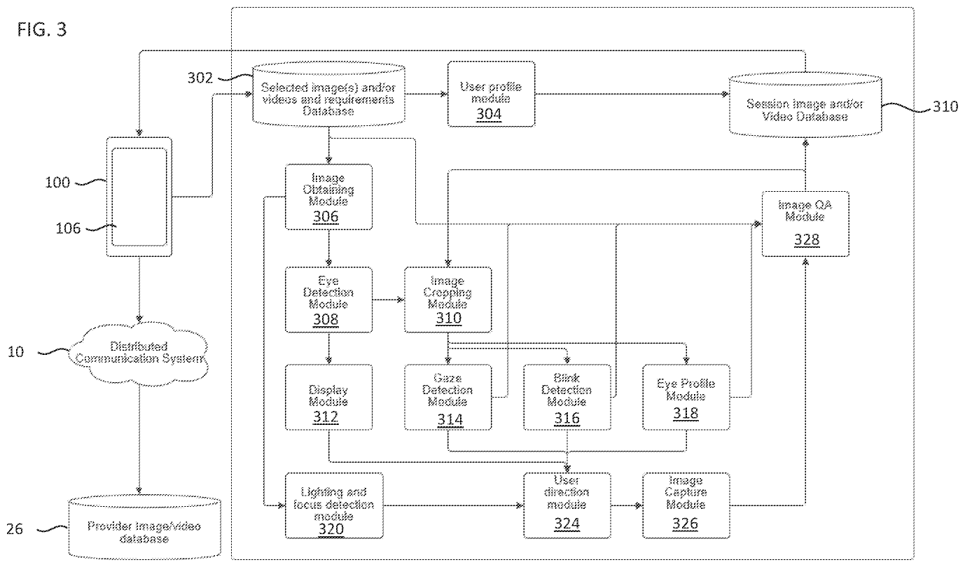

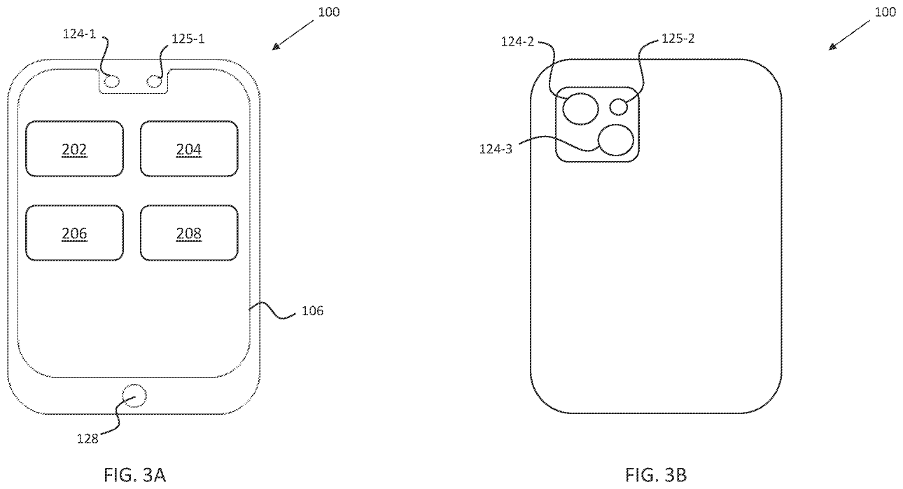

FIG. 3 illustrates one example of a processing system 300 that may be implemented by one or more processors 102 of a mobile device 100. Although FIG. 3 is described as being implemented by a mobile device 100, it should be understood that the processing system 300 may be implemented by other computing devices, such as computer 54, or in a distributed manner, such as in combination with multiple processing devices and/or in combination with telehealth network 20.

As shown in FIG. 3, the computing device 100 may include a database or other data storage 302 configured to store one or more selected images or sequence of images (e.g., video). The data storage may be stored in a main memory 104 and/or secondary memory 110 of the computing device, or the data storage may be stored remotely from the computing device 100. In some embodiments, the data storage 302 may also include one or more requirements, which may be predefined characteristics or parameters for one or more images or sequence of images that are to be obtained. For example, the characteristics and/or parameters may define a threshold or range of lighting requirements for an image, focus requirements, a size and/or location for a detected object and/or image, to list only a few possibilities. In some embodiments, the data storage 302 may include user identification and/or settings for the application being run on the mobile device. As will be understood by one of ordinary skill in the art, one or more tokens may be stored on the computing device 100 for gaining access to or authenticating with the telehealth network 20.

A user profile module 304 may be provided for facilitating registration of a user. In some embodiments, the user profile module 304 may be configured to present one or more GUIs to a user on display 106 to facilitate the registration of a user. Registration may include obtaining the user's name, password, gender, age, and other identifying characteristics of the user. In some embodiments, the user profile module 304 may query the user for screening purposes. For example, the questions presented to the user may inquire as to the types of services the user desires to obtain (e.g., general eye exam, contact lens fitting, glaucoma test, etc.) and/or whether the user is seeking services based on trauma, infection, or other reason. In some embodiments, the user profile module 304 may include logic for determining whether the user is part of a relevant population for receiving telehealth. For example, users of a certain age may be instructed to have an in-person appointment with a doctor for glaucoma screening, whereas the same user may be able to use the application for contact fitting. One of ordinary skill in the art will understand that the user profile module 304 may be configured to perform various other functions to facilitate registration and screening of a user, including the additional functions described below.

An image-obtaining module 306 may also be provided. The image-obtaining module 306 may be configured to cause an image or a series of images (e.g., a video) to be obtained using a camera 124. As described herein, the image-obtaining module 306 may be configured to cause the camera 124 to obtain one or more images automatically or in response to a user input, such as a voice command, selection of a soft button displayed on the display, or a press of a button, for example.

A separate detection module 308 may be provided or it may be combined into the image-obtaining module 306 (or vice versa). The detection module 308 may be configured to detect an object of interest, such as an eye, pupil, edge of a contact lens, or other object as will be understood by one of ordinary skill in the art. For example, the system described herein may be used for a wide variety of telehealth applications beyond those for optometry. In such implementations, the detection module 308 may be configured to detect other objects of interest, such as a tongue, epiglottis, or other parts of a patient's anatomy.

In some embodiments, detection module 308 may be implemented using a Yolov4-tiny ANN trained on a dataset of over 5,000 (e.g., 7,000) manually labeled images of various individuals to exclude biases. For example, the individuals may vary based on eye color, race, gender, and/or age, as will be understood by one of ordinary skill in the art. In some embodiments, the image resolution from the camera or image capture device is converted from a first size or resolution to a second size or resolution. For example, the images from the camera may be resized to have a resolution of 416×416, which may be a tradeoff between quality and processing speed. It should be understood that other resolutions may be used, and the images from the camera may not be resized before further processing. In some embodiments, the resizing of the image is performed by a separate module, such as image cropping module 310, as shown in FIG. 3. The image-cropping module 310 may be configured to crop an obtained image to a region of interest and/or size as determined by the detection module 308. The ANN may be converted from a first form to one or more second forms. For example, the ANN may be converted from a first form (e.g., a Darknet form), to a second or intermediate form (e.g., a Keras form), to a third or final form (e.g., a CoreML) so that the ANN may be run on a mobile device 100 and/or computer 54.

A display module 312 may be provided to work in communication with detection module 308. As will be understood by one of ordinary skill in the art, the display module 312 may be configured to interface with the display 106 of the computing device for presenting one or more captured images and/or GUIs to a user. In some embodiments, the display module 312 may be configured to display an overlay onto an image being displayed to a user. The overlay may take a variety of forms, such as a red line, circle, or polygon, and may be used to draw the user's attention to a region of interest in an obtained image. One of ordinary skill in the art will understand that the display module 312 may be configured to generate and/or display other overlays and/or GUIs for facilitating user interaction.

A gaze detection module 314 may be provided for detecting and/or tracking a gaze direction of a user. The gaze detection module 314 may be configured to analyze one or more images to detect an eye gaze direction. In some embodiments, the gaze detection module 314 may be configured to focus on a small region of the eye, which may be a region detected by the eye detection module 308 described above, and generate a binary mask for an iris (or other feature) of the eye.

The gaze detection module 314 may be implemented in a variety of ways. In some embodiments, the gaze detection module 314 may be implemented using a UNet neural network with a ReLU (rectified linear unit) activation function. However, it should be understood that the gaze detection module 314 may be implemented using other convolutional neural network for imaging. In some embodiments, the gaze detection module 314 may be trained on several hundred (e.g., 400 or more) pairs of eye iris region of interest segmentation masks, along with various augmentations to provide approximately 1,200 or more data points. In some embodiments, the image resolution from the camera or image capture device is converted from a first size or resolution to a second size or resolution. For example, the images from the camera may be resized to have a resolution of 192×128 and be scaled to fit a region of interest. It should be understood that other resolutions may be used, and the images from the camera may not be resized before further processing. In some embodiments, the gaze detection module 314 may receive an image that was previously cropped by the image cropping module 310 (or from detection module 308 if the cropping module 310 is included as a part of the detection module 308), as shown in FIG. 3.

A blink detection module 316 may also be provided for analyzing one or more images to determine when an eye is open or closed. In some embodiments, the blink detection module 316 is configured to detect a patient blinking in one or more images. For example, the blink detection module 316 may be configured to trigger the recording of a video (i.e., a series of images) where the beginning and ending of the recording is triggered by a blink, which may be identified by a transition between two states: an open eye and a closed eye. When the eye transitions from an eye open state to an eye closed state, the blink detection module may identify a blink.

In some embodiments, the blink detection module 316 may be configured to detect a blink when the eye is positioned from the image capture device by approximately 1 cm up to and including 30 cm. One of ordinary skill in the art will understand that the distances and ranges provided are merely for purposes of illustrating an example and may be based on the resolution of the image capture device and/or the processing power of the processor.

In some embodiments, the blink detection module 316 may be implemented using a Yolov4-tiny ANN trained on a dataset of 3,000 or more manually labeled images of various individuals having their eyes open and/or closed. For example, the individuals may vary based on eye color, race, gender, and/or age, as will be understood by one of ordinary skill in the art. In some embodiments, the image resolution from the camera or image capture device is converted from a first size or resolution to a second size or resolution. For example, the images from the camera may be resized to have a resolution of 416×416, which may be a tradeoff between image resolution and processing speed. It should be understood that other resolutions may be used, and the images from the camera may not be resized before further processing. The ANN may be converted from a first form to one or more second forms. For example, the ANN may be converted from a first form (e.g., a Darknet form), to a second or intermediate form (e.g., a Keras form), to a third or final form (e.g., a CoreML) so that the ANN may be run on a mobile device 100 and/or computer 54.

A profile module 318 also may be provided and may be configured to track a region of interest of the eye from a profile view in order to obtain images of an iris (or other feature) to facilitate the detection of glaucoma. As will be understood by one of ordinary skill in the art, glaucoma may result in fluid build-up in the eye, which can affect the optic nerve at the back (e.g. posterior) of the eye. One way in which glaucoma may be detected is through viewing a profile of the patient's eye and determining whether the iris is pushed forward (e.g., in an anterior direction) relative to a normal eye and/or whether fluid has built up in the anterior portion of the eye.

The profile-tracking module 318 may be implemented in a variety of ways. In some embodiments, the profile tracking module 318 may be implemented by a Yolov-tiny ANN pre-trained on an ImageNet dataset that is fine-tuned on a target dataset. For example, approximately 2,500 manually annotated profile images of a region of interest may be used to train the model, with different scales and data augmentation. Here again, to eliminate or reduce bias, the individuals may vary based on eye color, race, gender, and/or age, as will be understood by one of ordinary skill in the art.

In some embodiments, the image resolution from the camera or image capture device is converted from a first size or resolution to a second size or resolution. For example, the images from the camera may be resized to have a resolution of 416×416, which may be a tradeoff between quality and processing speed. It should be understood that other resolutions may be used, and the images from the camera may not be resized before further processing. The ANN may be converted from a first form to one or more second forms. For example, the ANN may be converted from a first form (e.g., a Darknet form), to a second or intermediate form (e.g., a Keras form), to a third or final form (e.g., a CoreML) so that the ANN may be run on a mobile device 100 and/or computer 54.

In some embodiments, a lighting and focus detection module 320 may be provided. As will be understood by one of ordinary skill in the art, the lighting and focus detection module 320 may be a separate module or integrated as part of another module, such as the image-obtaining module 306, for example. Further, the lighting and focus detection modules 320 may be implemented separately, such that there may be a standalone lighting detection and adjustment module and another separate focus detection and adjustment module. The lighting and focus detection module 320 may be configured to interface with the camera 12, such as to determine and/or adjust one or more of a lighting (e.g., flash) setting and a focus setting.

A user direction or instruction module 322 may be provided to interface with one or more of the display module 132, gaze detection module 314, blink detection module 316, eye profile module 318, and lighting focus and detection module 320. User detection module 322 may be configured with logic to receive one or more inputs from one or more other modules and determine one or more instructions to a user. For example, the user detection module 322 may be configured to receive an output from one or more other modules and determine whether corrective action is needed to be taken and thus instructions should be provided to a user. In one possible example, if a detected object (e.g., iris, pupil, eye) is found to be outside of the desired location and/or is not of a suitable size (e.g., too large or too small), then an instruction is generated and communicated to the user to take a corrective action. The instruction may be an audible instruction communicated to the user via a speaker 122, a message displayed on the display 106, and/or the mobile device may vibrate (e.g., by oscillator 123).

In some embodiments, an audible instruction may include a command or instruction for the user to move the camera in a specific direction in an attempt to obtain an image in which the object (e.g., eye, pupil, iris, or other desired object) is located in a suitable location in the image. For example, the command may instruct the user to move the camera to the left, right, up, and/or down to adjust the position of the user's face relative to the camera to change the location of the object in the image. Additionally or alternatively, the command may instruct the user to move the camera closer, farther away, and/or adjust a zoom setting in order to change the size of the object in the image. In some embodiments, the command may instruct the user to gaze in a different direction. For example, the user may be instructed to “Look Up,” “Look Down,” Look Left,” and/or “Look Right.” Other instructions may be provided for obtaining a video for purposes of assessing the fit of a contact lens. For example, instructions may be provided for the user to look forward and blink one eye at a time (e.g., “Look forward and blink your left eye. Continue looking forward and blink your right eye.”) followed by looking up and blinking one eye at a time (e.g., “Look up and blink your left eye. Continue looking up and blink your right eye.”).

In some embodiments, an instruction to move a camera relative to the user's eye (or the eye relative to the camera) may be displayed to the user, such as on a display. For example, the words “Move Camera Left,” “Move Camera Right,” “Move Camera Up,” “Move Camera Down,” “Move Camera Away,” and/or “Move Camera Closer” may be presented to a user on a display. In some embodiments, the display may be a display of a mobile device 100, although the display may be an auxiliary display that is separate from, but communicatively coupled to, the display of the mobile device. For example, the display may be a standalone display that displays a mirror image of the display 106 of the user device. In some embodiments, the rear-facing camera may be used and the user may be positioned in front of a mirror. The feedback may include displaying text on the display of the mobile device in a reversed fashion such that the user may be able to read the instructions/feedback in the mirror.

Additionally or alternatively, haptic feedback (e.g., instructions/commands) to the user may be provided. For example, one or a series of vibrations may be used to indicate where the camera should be moved relative to the user's eye (or vice versa). In some embodiments, one or more short vibrations and/or one or more long vibrations may be used to indicate a direction in which the camera should be moved. In some embodiments, the haptic feedback is direction such that the mobile device vibrates from a certain direction (e.g., from the left side of the mobile device) to indicate the direction in which the camera should be moved. One of ordinary skill in the art will understand that various types of instructions or commands may be implemented and conveyed to the user in a variety of ways, including through combinations of audible, visual, and/or tactile or haptic means.

Referring again to FIG. 3, an image capture module 326 may be provided for selecting one or more images for presenting to an image quality assurance (“QA”) module 328. In some embodiments, the image capture module 326 and the image QA module 328 may be combined into a single module or they may be implemented as separate modules as shown in FIG. 3. In some embodiments, the image capture module 326 is configured to receive one or more images from the user direction module 324 and forward to the image QA module 328. For example, the user direction module 324 may determine that no further instructions need to be provided to the user because an obtained image satisfies a preliminary set of criteria, such as suitable focus, lighting, and that an object within the obtain image meets certain criteria, such as size, location, and/or gaze.

The image capture module 326 may then provide one or more of these images to image QA module 328 to make a final determination as to which one or more images should be stored in a session image and/or video database 330. In some embodiments, the image QA module 328 may select a “best” image from a plurality of images for storage. For example, an image that best meets the one or more image requirements stored in the image/video requirements data store 302 may be selected from a plurality of images. The image QA module 328 may also provide feedback to one or more other modules, such as the image-cropping module 310, as shown in FIG. 3.

One of ordinary skill in the art will understand that the connections and general data flows shown in FIG. 3 merely are exemplary and additional and/or fewer connections and/or data flows may be implemented. For example, additional analysis modules may be provided, such as a module configured to provide a “digital stain” in an acquired image to alert a doctor or physician of a potential injury that the patient has suffered and been detected by the system. The digital staining may simulate fluorescein staining, which is used in many applications, including ophthalmic applications. For example, fluorescein is used as a diagnostic tool in the diagnosis of corneal abrasions, corneal ulcers, and herpetic corneal infections, as well as in rigid gas permeable contact lens fittings. The digital staining may be used in a similar manner by highlighting or identifying an area of interest or concern in the image.

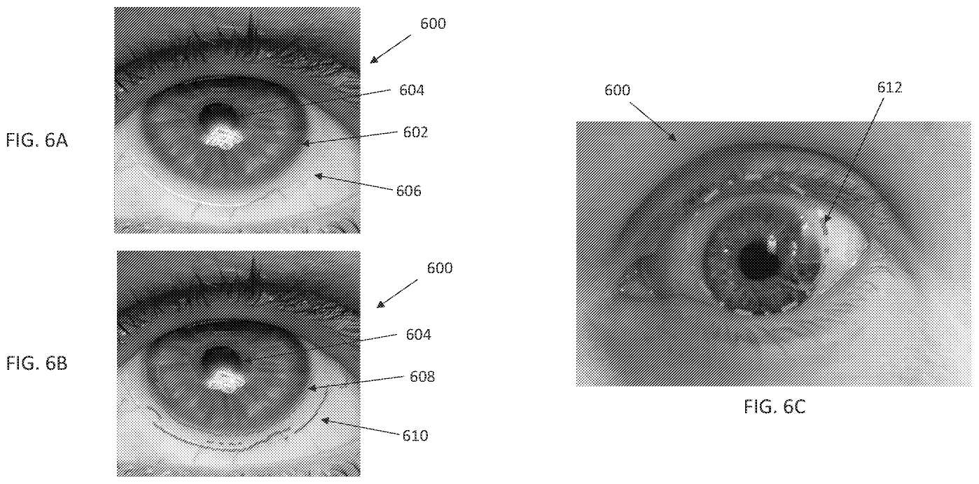

Further, as discussed above, the detection module may be configured to detect (and track) an edge of a contact lens (or other object of interest). In some embodiments, the edge of a contact lens may be tracked relative to one or more fixed landmarks, such as a boundary between an iris and a sclera, to assist an eye care provider in judging a fit of the contact lens. For example, when a patient tries on a new contact lens brand or prescription, a provider will often check to make sure that the contact lens is appropriate for the patient. The vertical motion of the lens, such as after the patient blinks, may provide the provider with valuable information as to whether the lens is appropriate for the patient. However, because contact lenses are typically clear, tracking the movement of a contact lens may be difficult. To assist a provider in performing such an assessment, in some embodiments, a standalone module may be provided for detecting such an object and/or providing an overlay or highlighting of the detected object in an acquired image. For example, FIG. 6A illustrates one example of an image or frame of an eye 600 having an iris 602 and a pupil 604. A contact lens 606 is shown as being disposed on the eye 600. FIG. 6B illustrates one example of the image or frame of the eye 600 with the edge of the iris 602 being identified with a first highlighting 608 and the edge of the contact lens 606 being identified with a second highlighting 610. FIG. 6C illustrates an example of an image of the eye 600 in which the outline of the contact lens 606 is highlighted 612 in a different manner. One of ordinary skill in the art will understand that various ways of highlighting an object of interest may be used. In some embodiments, an annotated video of a patient blinking may be generated and stored for review by a provider to assist in assessing the fit of a contact lens. It should be understood that the processing may also be performed in real-time and provided to the provider for assessing the fit of the contact lens in real-time via a network connection. In some embodiments, the information concerning the motion of the edge of the contact lens may also be processed to provide an automated judgment of the appropriateness of the lens for the user.

It should also be understood that the various modules of the processing pipeline shown in FIG. 3 may be performed on a mobile device 100 in real-time. As discussed herein, it should be understood that the processing may be implemented on devices with varying processing capabilities, including high-powered desktop computers and/or in a distributed cloud environment. In cloud computing examples, the images obtained by an image capture device or camera may be transmitted to one or more servers for processing, and the cloud and/or distributed processing system may provide one or more outputs from the processing to the mobile device 100 or computer 54.

Referring again to FIG. 1, telehealth network 20 may be accessible by patients and providers such that patients and providers may gain access to content stored in data storage units 26 of telehealth network 20. In some embodiments, patients and/or providers may gain access to the content using a computer 34, 54 and/or mobile device 100. For example, patients may download and install an application (e.g., an “app”) from a web store, such as Google Play, the Apple App Store, and/or the Microsoft Store, on a mobile device 100 and/or computer 54. The app, once installed, may facilitate access to the telehealth network 20. Additionally or alternatively, a patient may gain access to the telehealth network via a web page or a portal, such as the patient portal or interface 28, using a web browser. The access to the telehealth network may be protected and the patient user may need to enter a username and password to gain access. As will be understood by one of ordinary skill in the art, other ways of identifying the patient, such as through biometrics (e.g., facial recognition, fingerprints, eye scan) may be used. Such identification and authentication may be performed by a user profile module 304, as described above. A provider user (e.g., physician, clinician, and/or support staff) may also gain access to the telehealth network 20 by downloading and installing an app or via a provider portion or interface 30, as described above. As described below, the content accessible to a patient and/or a provider may differ.

Mobile devices typically are configured with multiple cameras, with the one or more cameras (e.g., camera 124-1) located on the front of the device, as shown in FIG. 3A, and one or more cameras located (e.g., cameras 124-2, 124-3) on the rear of the mobile device (i.e., the side of the mobile device opposite the screen), as shown in FIG. 3B. The cameras located on the rear of the device (e.g., those cameras 124-2, 124-3 shown in FIG. 3B) typically have greater resolution than the one or more cameras located on the front of the mobile device.

It also is possible for a mobile device 100 and/or computer 54 to be coupled to a camera that is separate from the mobile device 100 or computer 54, such as a peripheral camera that is coupled to the mobile device and/or computer 54 via a wireless or wired coupling. For example, the camera may be a high-resolution camera that is coupled to the mobile device 100 or computer 52 via a USB cord or via a network, such as a Wi-Fi network or a personal area network (e.g., Bluetooth). It may be desirable to use the camera with the greater available resolution in order to provide a physician or other medical provider with the most accurate and detailed images of the eye. However, given the fact that these high-resolution cameras may be located away from a display or screen, it may be difficult for the user to position the camera relative to the eye properly. Each of the various modules of the app installed on the user device (e.g., mobile device 100 and/or computer 54) or web application accessible via a network is configured to provide the user with guidance to ensure that one or more images obtained using a camera is located in a desired position in the frame. In some embodiments, the desired position is an approximate center of the frame of the acquired image.

Referring again to FIG. 3A, in some embodiments, one or more soft buttons 202, 204, 206, 208 may be displayed to the user on the display 106. In some embodiments, the soft generation and/or display of the soft buttons may be controlled by the user profile module 304 and/or the display module 312. The soft buttons may vary depending on whether the user is a patient or a provider. For example, a provider may be provided with one or more soft buttons that enable the provider to request one or more patients to perform one or more eye examinations using a mobile device 100 and/or computer 54.

In some embodiments, a provider may be presented with the ability to request a patient to obtain images of an anterior segment of an eye and/or to obtain one or more videos (e.g., a series of images) to be used to assess the fitting of one or more contact lenses. Obtaining one or more images of an anterior segment of the eye may including obtaining one or more images of one or more eyes, as well as one or more images of the eye when the eye is in different orientations and/or one or more images when the image capture device is located in different positions relative to the eye.

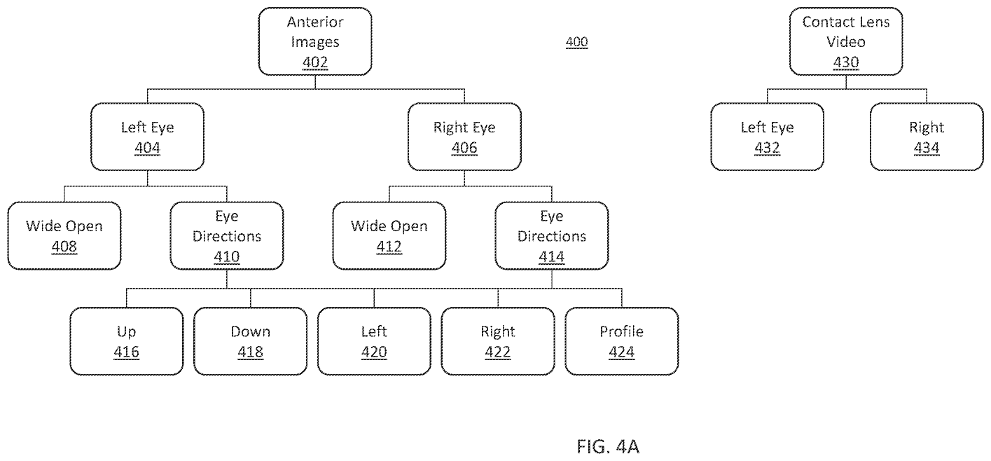

For example, FIG. 4A illustrates one example of a number of selections 400 that a provider user may be able to make via an app and/or web portal, such as provider portal or interface 30. As shown in FIG. 4A, a provider may be able to enter a request for a patient user to obtain one or more anterior images 402 or obtain a video of a contact lens fitting 430. If the provider user requests a patient user to obtain one or more anterior images 402, then the provider may be able to select the left eye 404 and/or right eye 406. For each of the left eye and right eye, a provider user may be able to select whether the patient user should obtain one or more images of the eye wide open 408, 412 and/or obtain one or more images of an eye in different orientations 410, 414 (e.g., looking up 416, looking down 418, looking left 420, looking right 422) and/or with a camera oriented to obtain a profile image 424. If the provider user requests a patient user to obtain one or more videos, then the provider user may also be able to select whether the user should obtain a video of the left eye 432 and/or right eye 434.

One of ordinary skill in the art that the optional selections illustrated in FIG. 4A are merely exemplary and fewer or additional selections may be implemented. Further, it should be appreciated that one or more GUIs may be presented to the provider user, such that the provider user may make the selections on a single screen or on multiple screens presented on a display 106. In some embodiments, the provider user may make the selections by selecting one or more of the soft buttons, e.g., soft buttons 202, 204, 206, 208, and/or through a voice input.

The instructions entered by a provider user may be communicated from a mobile device 100 and/or computer 54 to the telehealth network 20. In some embodiments, the provider instructions may be stored in a database or other data storage, such as one or more of the data storage units 26. The provider instructions may also be provided to a patient user. A patient user may be able to obtain the provided by the provider user by logging into the app running on a mobile device 100 and/or computer 54 and/or by logging into the patient portal 28.

Once a patient user logs into the app and/or web interface, the patient user may be presented with a number of soft buttons, such as one or more of the soft buttons 402, 404, 406, 408 illustrated in FIG. 3A. In some embodiments, the soft buttons presented to the patient user may be configured based on the provided instructions. For example, if the provider user provides instructions only for the patient to obtain anterior images of the left eye in a wide-open orientation, then a first set of soft buttons may be presented to the user on the display 106. If the provider user provides instructions for the patient to obtain both anterior images of both the left and right eyes wide open and in all directions, then a different set of soft buttons may be presented to the patient user. In some embodiments, the soft buttons may be the same regardless of the provider instructions, although some soft buttons may be greyed out or inactive if the provider does not provide instructions to obtain certain images. One of ordinary skill in the art will understand that the manner in which the soft buttons and general architecture of the user interface and one or more GUIs may be configurable.

FIG. 4B illustrates one example of a number of selections 450 that may be available to a patient user. As shown in FIG. 4B, a patient user may be prompted to obtain anterior images of an eye 452 and/or a video of a contact lens positioned in an eye 480. In some embodiments, the patient user may be able to select the order in which the one or more images and/or video are obtained, such as by pressing one of the soft buttons or by providing a voice input, as will be understood by one of ordinary skill in the art.

If the user selects the anterior images 452, the user may be given the option to obtain one or more images of the left eye 454 and/or the right 456. In some embodiments, the user may be instructed or prompted to obtain the images in a certain, predetermined order (e.g., left first, then right). Similarly, the patient user may be able to select whether to obtain one or more images of an eye in a wide open position 458, 462 and/or to obtain one or more images in which the eye is looking in one or more directions 462, 464 (e.g., looking up 466, looking down 468, looking left 470, looking right 472) or the camera is position to obtain a profile image 474. Alternatively, the user may be instructed or prompted to obtain the images in a certain, predetermined order, which may vary, as will be understood by one of ordinary skill in the art.

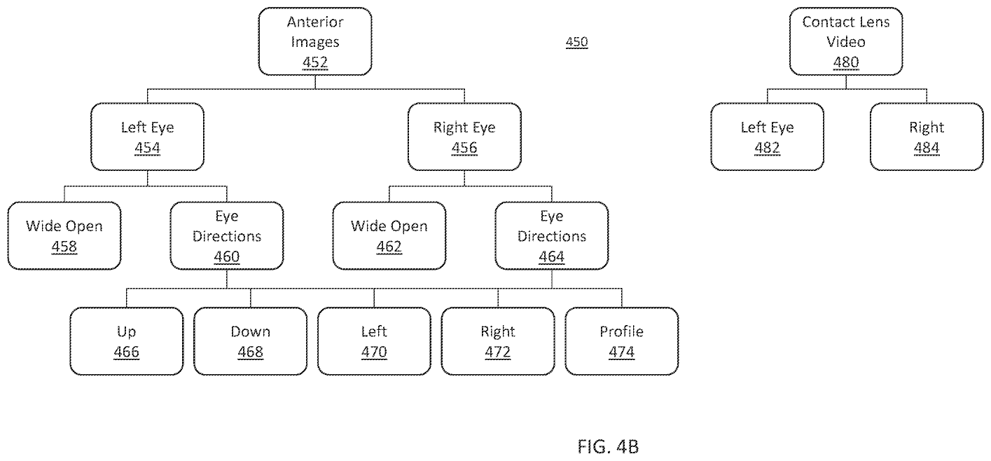

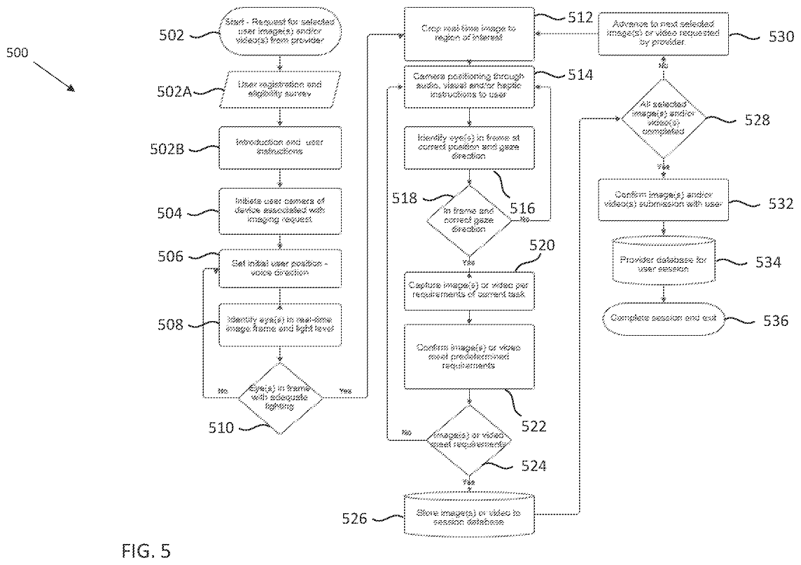

FIG. 5 illustrates one example of a process flow that may be performed to obtain one or more images and/or videos in accordance with some embodiments. At block 502, the process begins. As noted above with respect to FIGS. 4A and 4B, the process may begin in response to a user selecting one or more images and/or videos to have obtained, or the process may begin automatically with the user being instructed to obtain a specific image and/or video. In some embodiments, the user may be prompted to register and/or screened before being instructed to obtain one or more images, as block 502A. Additionally or alternatively, the user may also be presented with a tutorial or other instructions at block 502B. In some embodiments, the processes performed at blocks 502, 502A, 502B may be implemented by the user profile module 304 and/or image obtaining module 306

At block 504, a camera 124 or other image capture device is initialized. The initialized camera may be a front-facing camera (e.g., camera 124-1 shown in FIG. 3A), a rear-facing camera (e.g., camera 124-2, 124-3 shown in FIG. 3B), or a peripheral camera. In some embodiments, the image-obtaining module 306 may initialize the image capture device.

At block 506, the user may be instructed to prepare for one or more images to be obtained. For example, the image obtaining module 306 and/or user direction module 324 may be configured to provide instructions to the user to position the image capture device relative to the user and/or position the user relative to the image capture device to obtain an image of a first type. In one example, the user may be instructed to position the image capture device so that it is parallel to a frontal or sagittal of the user with the image capture device aimed at area of interest (e.g., face, head, eye, or other part of the body that may be of interest). The instructions may be provided to the user audibly, visibly, and/or haptically and may be based on a predetermined order of images and/or videos to be collected, such as determined from instructions received from a provider or input by the user.

At block 508, a first set of one or more images are obtained and an area or object of interest may be determined. In some embodiments, the first set of one or more images may be obtained in response to a user selection, such as the user pressing a physical button (e.g., input button 128) and/or a virtual or soft button. In some embodiments, the first set of one or more images may be obtained in response to the user speaking a command, which audible command or utterance may be detected by microphone 128 in combination with an audio detect process executed by the one or more processors 102, as will be understood by one of ordinary skill in the art. In some embodiments, the first set of one or more images may be obtained automatically after a predetermined amount of time elapses. For example, a countdown may be communicated to the user audibly, visually, and/or tactically, and then the first set of one or more images may be acquired by the initialized camera. The audible countdown may be emitted from a microphone 126, the visual countdown may be displayed on a display 106, and a tactical countdown may be provided by an oscillator 123. In some embodiments, the first set of one or more images may be obtained automatically without providing the user with an indication that the first set of one or more images is to be taken. For example, once the camera is initialized, the first set of one or more images may automatically be acquired by the camera.

In some embodiments, a burst of images may be obtained by the image capture device 124, which may be controlled by the image-obtaining module 308, and the burst of images are analyzed in real-time by the detection module 308. As described above, the detection module 308 may include an ANN configured to detect an object, such as an eye or pupil, in an image. Further, the lighting and focus detection module 320 may be configured to analyze the burst of images and determine whether the lighting and/or focus of the obtained images is acceptable. Although the modules are described as operating on a burst of images (e.g., two or more), it should be understood that the modules may be configured to operate on a single image.

At block 510, a decision is made as to whether the object (e.g., eye(s), pupil(s) or other object that is to be imaged) is located in an image frame with adequate lighting and focus. In some embodiments, the analysis performed at blocks 508 and/or 510 also includes determining whether the location of the object is suitable includes determining a location of the object in a frame. For example, a distance between an edge of the object and a reference point, such as an edge and/or center of the image, may be determined. If the distance is outside a predetermined range (e.g., too far from a center) or within a certain range (e.g., too close to an edge of the frame), then it may be determined that the location of the object in the first set of one or more images is not suitable. Additionally or alternatively, a size of the object may be determined and compared to a predetermined size or other criterion. In some embodiments, a size of the object may be based on a number of pixels in which the object is detected. One of ordinary skill in the art will understand that there may be other ways of determining a size of the object, such as determining a number of pixels in which the object is not detected. In some examples, the user may be prompted to provide a specific reference object having a known size (e.g., credit card, coin) that is used to determine the sizes of other objects in the image. In some examples, an assumption about the size of an anatomical landmark in the image may be made. The assumption of the landmark (e.g., iris diameter) may be average size of the landmark for all people, or the assumption may be based on the user (e.g., gender, age, race, or other attributes of the user). In some examples, a sensing camera, such as a “TrueDepth” camera available on iPhones may be used to measure a distance between the camera and the user. The distance, when combined with the camera's focal length, may be used to determine a conversion between image pixels and physical units, such as millimeters. Other examples of determining size of an object are disclosed in U.S. Pat. No. 9,532,709 and U.S. Patent Application Publication No. 2021/0065285, which are incorporated by reference in their entireties. The decision at block 510 may be made by one or more (e.g., combinations) of the detection module 308, the lighting and focus detection module 320, and the user direction module 324.

If a determination is made that the first set of one or more image(s) are not suitable, e.g., an object is not in the frame (or is not in a desired area of the frame), the object is too small or too large, the lighting is too low or too bright, the image is out of focus, then the process may move back to block 506 wherein the user direction module 324 and/or lighting and focus correction module 320 may provide the user to take a corrective action. For example, if the detected object is found to be outside of the desired location, is not of a suitable size (e.g., too large or too small), is of poor lighting and/or focus, then an instruction is generated and communicated to the user to take a corrective action. The instruction may be an audible instruction communicated to the user via a speaker 122, a message displayed on the display 106, and/or the mobile device may vibrate (e.g., by oscillator 123).

An audible instruction may include a command or instruction for the user to move the camera in a specific direction in an attempt to obtain a second set of one or more images in which the object (e.g., eye, pupil, iris, or other desired object) is located in a suitable location in the frame(s) of the image(s). For example, the command may instruct the user to move the camera to the left, right, up, and/or down to adjust the position of the user's face relative to the camera to change the location of the object in the image. Additionally or alternatively, the command may instruct the user to move the camera closer, farther away, and/or adjust a zoom setting in order to change the size of the object in the frame. In some embodiments, the command may instruct the user to gaze in a different direction. For example, the user may be instructed to “Look Up,” “Look Down,” Look Left,” and/or “Look Right.

As noted above, in some embodiments, an instruction to move a camera relative to the user's eye (or the eye relative to the camera) may be displayed to the user, such as on a display. For example, the words “Move Camera Left,” “Move Camera Right,” “Move Camera Up,” “Move Camera Down,” “Move Camera Away,” and/or “Move Camera Closer” may be presented to a user on a display. In some embodiments, the display may be a display of a mobile device 100, although the display may be an auxiliary display that is separate from, but communicatively coupled to, the display of the mobile device. For example, the display may be a standalone display that displays a mirror image of the display 106 of the user device.

Additionally or alternatively, haptic feedback (e.g., instructions/commands) to the user may be provided. For example, one or a series of vibrations may be used to indicate where the camera should be moved relative to the user's eye (or vice versa). In some embodiments, one or more short vibrations and/or one or more long vibrations may be used to indicate a direction in which the camera should be moved. In some embodiments, the haptic feedback is direction such that the mobile device vibrates from a certain direction (e.g., from the left side of the mobile device) to indicate the direction in which the camera should be moved. One of ordinary skill in the art will understand that various types of instructions or commands may be implemented and conveyed to the user in a variety of ways, including through combinations of audible, visual, and/or tactile or haptic means.

The process may then proceed to blocks 508 and 510 where an additional set of one or more images may be obtained and analyzed as described above. In this manner, the process may perform a continuous loop until an acceptable set of one or more images has been obtained.

Once a determination is made that a set of one or more image(s) are suitable or acceptable, then the process may move to block 512 where the acceptable set of one or more images are cropped to a first region of interest. In some embodiments, the cropping of the acceptable set of one or more images may be performed in real-time (e.g., operating on the images as they are received from the camera with introducing additional delay beyond the delay attributed to the processing) by the image cropping module 310. For example, the acceptable set of one or more images may be cropped to reduce the size of the overall image to an area that excludes irrelevant features and includes a specific region of interest, such as an eye, pupil, or other features or objects.

At block 514, the user direction module 320 may generate one or more instructions or prompts for the user to move the image capture device and/or the user's body to collect a second set of one or more images and/or video. In some embodiments, the instructions provided to the user for obtaining a second set of one or more images and/or video are based on the request for the image(s) and/or video(s) at block 502. The instructions generated by the user direction module 320 at block 514 may be generated and/or communicated to the user in a similar manner as those described above with respect to block 506. In some embodiments, the user may be properly positioned already and the instructions may request the user to stay still or maintain a position.

At block 516, an object is identified in a frame and an orientation is determined. For example, if the object of interest is an eye or pupil, then the frame may be analyzed to ensure that the pupil or eye is within the frame and is gazing in a desired direction (e.g., straight, up, down, left, right, etc.). The analysis at block 516 may be performed by one or more of the detection module 308, cropping module 310, and the gaze detection module 314. It should be understood that the process may be used to analyze other objects or body parts that are amenable to telehealth imaging, including ears, nose, and throats, to list only a few possibilities. Consequently, the object being detected may be a tongue and the orientation may be an extended position (e.g., sticking out of the mouth). One of ordinary skill in the art will understand that other objects and orientations may be determined.

At block 518, a decision is made as to whether the object has been properly detected and is oriented correctly. In some embodiments, the determination is made based on the analysis performed at block 516 by one or more of the detection module 308, cropping module 310, and the gaze detection module 314. If the object is not in frame and/or is not in the proper orientation, then the process may proceed back to block 514 where one or more instructions may be generated by the user direction module 320. As will be understood by one of ordinary skill in the art, the user direction module 320 may receive, as an input, the output of the analysis performed by the one or more of the detection module 308, cropping module 310, and the gaze detection module 314 and generate an appropriate instruction for the user to correct the issue. The one or more instructions may be similar to those described above with respect to block 506, and the process may then proceed to blocks 516 and 518. Repetitive descriptions of the instructions, analysis, and decisions made at blocks 506, 516, 518 are not provided. In this manner, the process may perform a continuous loop until the object is located in the frame and in the desired orientation.

Once a determination at block 518 is made that the objection is in frame and properly oriented, then the process may move to block 520. At block 520, one or more images and/or video are captured for further processing. For example, the image capture module 326 may select one or more of the images in which the object is located in frame and in a proper orientation for further analysis.

At block 522, the one or more images selected at block 520 are analyzed by the image QA module 328, which also may utilize one or more of the gaze detection module 314, blink detection module 316, and eye profile module 318. For example, the image QA module 328 may analyze the selected one or more images to make a determination at block 524 as to whether the selected images meet a set of predetermined requirements. In some embodiments, the set of predetermined requirements may be stored in the image/video requirements database 302 described above. In some embodiments, the image QA module may consult the output of one or more of the gaze detection module 314, blink detection module 316, and eye profile module 318 to ascertain whether the selected images meet these requirements and identify one or more of the one or more selected images that best meets these requirements.

If, at decision block 524, it is determined that none of the selected images meet the requirements, then the process may move back to block 514 where one or more instructions may be generated by the user direction module 320 as described above. The one or more instructions may be similar to those described above with respect to block 506, and the process may then proceed through the process described above with respect to blocks 516, 518, 520, 522. Repetitive descriptions of the instructions, analysis, and decisions made at blocks 506, 516, 518, 520, 522 are not provided. In this manner, the process may perform a continuous loop until one or more images or video meets the requirements.

Once one or more images and/or video is identified as meeting the requirements, then the one or more video and/or images may be stored in the session image/video database 310 at block 526. As described above, the one or more images and/or videos may be stored locally on the mobile device 100 or computer 54. However, in some embodiments, the one or more images and/or video may be stored in a remote computer readable storage medium, such as a computer readable storage medium located in the telehealth network 20 (e.g., in a data storage unit 26). As will be understood by one of ordinary skill in the art, the image (or a copy of the image) may be transmitted from the mobile device 100 or computer 54 to the telehealth network for storage. The image may be and/or stored in an encrypted form in a database (e.g., a relational database) and/or other data storage, such as a simple storage service (S3) object store. In some embodiments, the stored image may be associated with a unique identifier for the patient user to facilitate retrieval of the image, such as by a physician or other practitioner.

At decision block 528, a determination is made as to whether an additional image and/or video is needed. In some embodiments, the determination is made based on the type of eye exam that is selected by the user and/or prescribed by the physician, as described above. For example, a particular eye exam may be configured to collect several images of an eye, such as an image in which the eye is looking directly at the camera (e.g., straight ahead), an image in which the eye is looking up, an image in which the eye is looking down, an image in which the eye is looking left, and/or an image in which the eye is looking right. In some embodiments, the eye examination may require one or more images to be obtained of both eyes of a person. In such examples, the program will track which images have been obtained and stored and which images have not yet been obtained. Accordingly, if an image of an object in a specific orientation or position has not yet been obtained then control of the process will move to block 530.

At block 530, another set of parameters and instructions are generated for obtaining another image and/or video. For example, if the first image that was successfully obtained and stored at block 526 was an image of eye staring straight ahead and the user is to obtain an image of the eye looking in another direction or when the camera is arranged to obtain a profile view of the eye, then the instructions may be loaded and provided to the user direction module, user profile module, and/or image obtaining module. The user may then be instructed to reposition the image capture device and/or the user for obtaining the next desired image and/or video. In some embodiments, the process may move to block 512 where the images being obtained by the image capture device are segmented to determine how the user should reorient the image capture device and/or image capture device for obtaining the next desired image or video. The process may then proceed through blocks 512-528 as described above.

If at decision block 528 it is determined that all images and/or video have been obtained, then the process may move to block 532. In some embodiments, the user may be presented with the one or more images and/or videos captured during the session by the user profile module 304. The user may be given the ability to submit a replacement (e.g., retake) image for one or more of the acquired and stored images prior to the images being submitted to the telehealth network 20. If the user decides to retake or replace an image, then the process may proceed back to block 504 or block 512. In some embodiments, the user may be prompted, such as with a GUI and/or soft button, to confirm the submission to the telehealth network 20. Once the user approves the submission of the one or more images or videos, then the process may move to block 534.

At block 534, the one or more acquired images and/or video may be stored in response to user input at block 532. As described above, the one or more acquired images and/or video may be stored in a computer readable storage medium that is remotely located from mobile device 110 or computer 54, such as a computer readable storage medium located in the telehealth network 20 (e.g., in a data storage unit 26). As will be understood by one of ordinary skill in the art, the image (or a copy of the image) may be transmitted from the mobile device 100 or computer 54 to the telehealth network 20 for storage. The image may be and/or stored in an encrypted form in a database (e.g., a relational database) and/or other data storage, such as a simple storage service (S3) object store. In some embodiments, the stored image may be associated with a unique identifier for the patient user to facilitate retrieval of the image, such as by a physician or other practitioner.

At block 536, the process ends. In some embodiments, the user is automatically logged out of the session once the images and/or video have been transmitted to the remotely located computer readable storage medium (and receipt of such images and/or video has been confirmed) or the user may be prompted to terminate the session, such as by pressing a soft button or through other means as will be understood by one of ordinary skill in the art.

The disclosed systems and methods may be embodied in the form of program code embodied in tangible media, such as floppy diskettes, CD-ROMs, DVD-ROMs, Blu-ray disks, hard drives, or any other machine-readable storage medium, wherein, when the program code is loaded into and executed by a machine, such as a computer, the machine becomes an apparatus for practicing the method. The present systems and methods may also be embodied in the form of program code, for example, whether stored in a storage medium, loaded into and/or executed by a machine, or transmitted over some transmission medium, such as over electrical wiring or cabling, through fiber optics, or via electromagnetic radiation, wherein, when the program code is loaded into and executed by a machine, such as a computer, the machine becomes an apparatus for practicing the method. When implemented on a general-purpose processor, the program code segments combine with the processor to provide a unique device that operates analogously to specific logic circuits.

Although the devices, systems, and methods have been described in terms of exemplary embodiments, they are not limited thereto. Rather, the appended claims should be construed broadly, to include other variants and embodiments of the devices, systems, and methods, which may be made by those skilled in the art without departing from the scope and range of equivalents of the devices, systems, and methods.

What is claimed is:

1. A method, comprising:
determining, by a first processing module, if an object is present in a first image received from an image capture device;
if the object is determined to be present in the first image, then determining, by a second processing module, if the object is meets at least one predetermined criterion;
if the object does not meet the at least one predetermined criterion, then determining, by a third processing module, at least one adjustment to be made; and
communicating an instruction to perform the at least one adjustment,
wherein the at least one predetermined criterion includes at least one of a size and a location in an image.

2. The method of claim 1, wherein the first, second, and third processors are the same processor.

3. The method of claim 1, wherein the first, second, and third processors are different processors.

4. The method of claim 1, wherein the instruction to perform the at least one adjustment is communicated to a user using an audio output device.

5. The method of claim 1, wherein the instruction to perform the at least one adjustment is communicated to a user haptically.

6. The method of claim 1, wherein the instruction to perform the at least one adjustment is displayed to a user.

7. The method of claim 1, further comprising providing an indication that the at least one predetermined criterion has been met.

8. The method of claim 7, wherein the indication includes at least one of a haptic indication, a visual indication, or an audible indication.

9. The method of claim 7, wherein the indication includes an instruction to acquire a second image.

10. A machine-readable storage medium storing executable code, the executable code, when executed by a processor, causes the processor to perform a method, the method comprising:
receiving a first image obtained by an image capture device;
determining if a first object is in the first image;
determining if a size and a location of the first object in the first image meet threshold values if the first object is determined to be in the first image;
determining at least one corrective action to be taken if at least one of the size and the location of the first object in the image does not meet the threshold values; and
causing an instruction for taking the at least one corrective action to be communicated to a user.

11. The machine-readable storage medium of claim 10, wherein the first object is at least one of an iris and a pupil of an eye.

12. The machine readable-storage medium of claim 10, wherein the first object is a contact lens.

13. The machine-readable storage medium of claim 10, wherein the at least one corrective action includes moving the image capture device in at least one direction relative to at least one of an eye of a user and the object.

14. The machine-readable storage medium of claim 13, wherein the image capture device is a camera located on a side of a mobile device that is opposite a side on which a screen is disposed.

15. The machine-readable storage medium of claim 10, wherein the at least one corrective action includes moving an eye of a user relative to the image capture device.

16. The machine-readable storage medium of claim 10, wherein determining if the first object is in the first image includes using a first neural network.

17. The machine-readable storage medium of claim 16, wherein the method includes tracking the first object in at least one second image obtained from the image capture device using a second neural network that is different from the first neural network.

18. The machine-readable storage medium of claim 10, wherein the method includes storing the first image in a non-transient machine readable storage medium that is communicatively coupled to the processor if the size and the location of the first object in the first image meet threshold values.

19. The machine-readable storage medium of claim 10, wherein the method includes analyzing a plurality of images received from the image capture device and determine a blink of an eye.

20. The machine-readable storage medium of claim 10, wherein the instruction for taking at least one corrective action is communicated to the user by at least one of an audio output device, a haptic device, or on a display device.

21. The machine-readable storage medium of claim 10, wherein the method includes providing an indication that the at least one of the size and the location of the first object in the image meets the threshold values.

22. The machine-readable storage medium of claim 21, wherein the indication includes at least one of a haptic indication, a visual indication, or an audible indication.

23. The machine-readable storage medium of claim 21, wherein the indication includes an instruction to acquire a second image.
